(19) United States
(12) Patent Application Publication
(10) Pub. No.: US 2017/0227519 A1
(43) Pub. Date: Aug. 10, 2017

(54) OPTICAL VIEWING DEVICE

(71) Applicant: Aidmics Biotechnology Co., Ltd., Taipei City (TW)

(72) Inventors: Cheng-Ming LIN, Taipei City (TW); Chang-Yu CHEN, Taipei City (TW); Tsun-Chao CHIANG, Taipei City (TW); Shu-Sheng LIN, Taipei City (TW)

(21) Appl. No.: 15/411,512

(22) Filed: Jan. 20, 2017

(30) Foreign Application Priority Data
Feb. 5, 2016 (TW) 105104130

(51) Int. Cl. G01N 33/487; G02B 21/06; G02B 21/36

(52) U.S. Cl. CPC G01N 33/487; G02B 21/36; G02B 21/06

(57) ABSTRACT

An optical viewing device includes a first assembly and a second assembly. The first assembly includes a first body and a light source. The second assembly includes a second body, a convex lens and an image capturing module. The first body has a first connecting portion and a light output hole, and the first connecting portion is disposed on at least one side of the light output hole. The light source is disposed within the first body. The second body has a second connecting portion, which is disposed corresponding to the first connecting portion. The convex lens and the image capturing module are disposed within the second body, and the light outputted from the light output hole passes through the convex lens and then enters the image capturing module. The first connecting portion and the second connecting portion are connected with each other to form a chamber.

### CROSS REFERENCE TO RELATED APPLICATIONS

This Non-provisional application claims priority under 35 U.S.C. §119(a) on Patent Application No(s). 105104130 filed in Taiwan, Republic of China on Feb. 5, 2016, the entire contents of which are hereby incorporated by reference.

### BACKGROUND OF THE INVENTION

Field of Invention

This invention relates to an optical viewing device and, in particular, to a portable optical viewing device.

Related Art

The artificial insemination (AI) technology can effectively use superior genetic factor, simplify breeding operation, reduce pathophoresis and lower down the cost of managing boar breeding, so as to have irreplaceable importance in the swine breeding industry. The vitality of the boar spermatozoon is usually viewed by a phase-contrast microscope, and the mobility of the whole spermatozoon of the semen sample is represented in percentage. The original spermatozoon freshly collected must have a concentration of 80%˜85% or higher. The pre-mixed original spermatozoon that has passed through the detection can be diluted to a required concentration in multiple by a thinner.

However, a conventional detecting apparatus, such as a microscope and a cell counter, is a big burden for pig farmers due to its high price. Purchasing a detecting apparatus just for the detection is not cost-effective. Besides, the detecting apparatus is not easily taken to every piggery for the detection due to its volume and weight, so that it is not suitable for pig farmers to have a collective purchase and use. Besides, the detection conducted by using the conventional detecting apparatus needs to be performed by the professional operator, so that the detected samples are usually conveyed with the low-temperature preservation to the detecting station for the detection. Therefore, the pig farmers need to wait for several days to obtain the detection result, and besides, the conveying procedure and the temperature variation during the conveying process will further influence the vitality and fresh level of the sample.

Since the technology is improved a lot, the existing mobile communication devices, such as cell phones or tablet computers, not only have good portability but also are equipped with a certain level of computing capability so that they can process some easy computation which only could be performed by the computer of a laboratory before. Therefore, in comparison with the conventional way, there is a need to present a kind of detecting apparatus which has a lower price and good portability and can be used in cooperation with the cell phone or tablet computer, so that the pig farmers can afford to buy the detecting apparatus and take it to the sampling site to do a direct detection by themselves. Thus, the detecting process can be simplified, and besides, the pig farmers needn't wait for several days to obtain the detection result and can make sure the viewed sample is the freshest.

### SUMMARY OF THE INVENTION

In view of the foregoing subject, an objective is to provide an optical viewing device which is portable and can be used in cooperation with the cell phone or tablet computer, wherein two components are easily assembled to preserve the sample therein for the observation and the viewed microscopic image can be transmitted to the cell phone or the tablet computer for the computation. Therefore, the threshold of the detection of the biological sample can be lowered down a lot, so that the user untrained professionally can still rapidly perform simple and easy biological sample detection everywhere.

To achieve the above objective, the present invention discloses an optical viewing device, which is cooperated with an external image device and includes a first assembly and a second assembly. The first assembly includes a first body and a light source, while the second assembly includes a second body, a convex lens, an image capturing module and a communication module. The image capturing module catches the image formed through the convex lens and generates a digital image accordingly. The communication module transmits the digital image signal to an external electronic device or a cloud server for later processes, analyzing, storing or displaying.

In the optical viewing device of the present invention, the first body includes a first connecting portion and a light output hole. The first connecting portion is disposed on at least one side of the light output hole. The light source is disposed within the first body. The second body includes a second connecting portion, which is disposed corresponding to the first connecting portion. The convex lens and the image capturing module are disposed within the second body. The light outputted from the light output hole passes through the convex lens and then enters the image capturing module. The first connecting portion and the second connecting portion match and connect each other to fix and combine the first and second bodies and form a chamber.

In another embodiment, the first body includes a first connecting element and a light output hole. The first connecting element is disposed on at least one side of the light output hole. The light source is disposed within the first body. The second body includes a second connecting element, which is disposed corresponding to the first connecting element. The convex lens and the image capturing module are disposed within the second body. The first connecting element and the second connecting element match and connect each other to fix and combine the first and second bodies and form a chamber.

In one embodiment, the first connecting portion/element is disposed around the light output hole.

In one embodiment, the second body includes a light input hole, and the second connecting portion/element is disposed around the light input hole. The light emitted by the light source sequentially passes through the light output hole, the chamber and the light input hole.

In one embodiment, the first connecting portion and the second connecting portion are connected to each other by screw or tenon.

In one embodiment, the first connecting element and the second connecting element are connected to each other by locking or magnetic attraction.

In one embodiment, the chamber includes an opening, which matches a carrier.

In one embodiment, the chamber includes an opening disposed on the first body or the second body, and the opening matches a carrier.

In one embodiment, the first body comprises a first breach, the second body comprises a second breach, and the first breach and the second breach match and connect each other to form the opening of the chamber, and the opening matches a carrier.

In one embodiment, the first assembly further includes a light guiding element disposed at the light output hole. The light emitted by the light source sequentially passes through the light output hole, the light guiding element, the chamber and the light input hole.

In one embodiment, the first assembly further includes a light guiding element and a sampling assembly covering the light guiding element. The light guiding element is disposed at the light output hole, and the light emitted by the light source sequentially passes through the light output hole, the light guiding element, the chamber and the light input hole.

In one embodiment, the first assembly further includes a light guiding element and a sampling assembly covering the light guiding element. The light guiding element is disposed at the light output hole, and the light emitted by the light source sequentially passes through the light output hole, the light guiding element, the chamber and the light input hole. The sampling assembly includes a first cup element covering the light guiding element and a second cup element covering an outside of the first cup element. The first cup element and the second cup element are connected to each other in a concave-convex match to form a sample containing space.

In one embodiment, the first assembly further includes a light guiding element and a sampling assembly covering the light guiding element. The light guiding element is disposed at the light output hole, and the light emitted by the light source sequentially passes through the light output hole, the light guiding element, the chamber and the light input hole. The sampling assembly includes a first cup element covering the light guiding element, a second cup element covering an outside of the first cup element, and at least a positioning portion. The first cup element and the second cup element are connected to each other in a concave-convex match to form a sample containing space, and the positioning portion is disposed in the sample containing space.

In one embodiment, the first assembly further includes a light guiding element and a sampling assembly covering the light guiding element. The light guiding element is disposed at the light output hole, and the light emitted by the light source sequentially passes through the light output hole, the light guiding element, the chamber and the light input hole. The sampling assembly includes a first cup element covering the light guiding element, a second cup element covering an outside of the first cup element, and at least a positioning portion. The first cup element and the second cup element are connected to each other in a concave-convex match to form a sample containing space. The positioning portion is disposed in the sample containing space and is connected with one of the first and second cup elements.

As mentioned above, the optical viewing device of the invention can be used in cooperation with the cell phone or tablet computer, and the two components are easily assembled to preserve the sample therein for the observation. Moreover, the viewed microscopic image can be transmitted to the cell phone or the tablet computer for the computation. Therefore, the threshold of the detection of the biological sample can be lowered down a lot, so that the user untrained professionally can still rapidly perform simple and easy biological sample detection everywhere.

### BRIEF DESCRIPTION OF THE DRAWINGS

The invention will become more fully understood from the detailed description and accompanying drawings, which are given for illustration only, and thus are not limitative of the present invention, and wherein:

FIG. 1A is a schematic diagram showing the appearance of the assembled optical viewing device of an embodiment of the invention;

FIG. 1B is a schematic diagram of the exploded perspective view of the optical viewing device in FIG. 1A;

FIG. 2A is a schematic sectional diagram of the optical viewing device of FIG. 1A taken along the line A-A;

FIGS. 2B to 2E are schematic diagrams of different embodiments of the optical viewing device of FIG. 2A;

FIG. 3 is a schematic diagram showing the appearance of the first assembly of the optical viewing device of another embodiment of the invention;

FIG. 4 is a schematic diagram showing the appearance of the first assembly of the optical viewing device of another embodiment of the invention;

FIG. 5 is a schematic diagram showing the appearance of the assembled optical viewing device of another embodiment of the invention;

FIG. 6 is a schematic exploded diagram of the optical viewing device in FIG. 5;

FIGS. 7A and 7B are schematic sectional diagrams of the optical viewing device of FIG. 5 taken along the line A-A;

FIG. 8 is a schematic diagram showing the appearance of the optical viewing device of another embodiment of the invention;

FIG. 9 is a schematic exploded diagram of the optical viewing device in FIG. 8;

FIG. 10 is a schematic exploded diagram of another embodiment of the optical viewing device in FIG. 8; and

FIG. 11 is a schematic exploded diagram of another embodiment of the optical viewing device in FIG. 8.

### DETAILED DESCRIPTION OF THE INVENTION

The present invention will be apparent from the following detailed description, which proceeds with reference to the accompanying drawings, wherein the same references relate to the same elements.

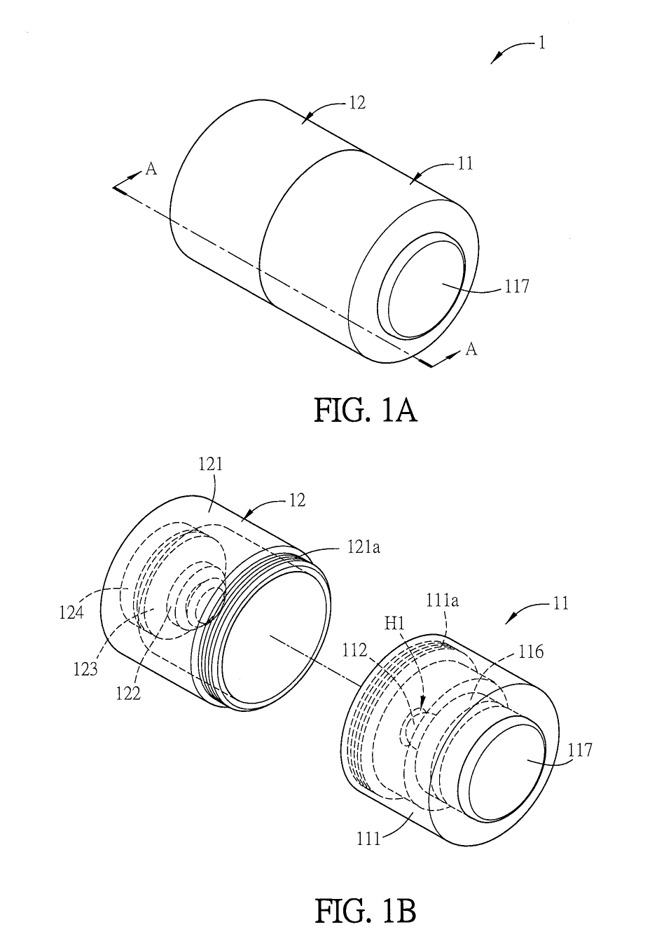

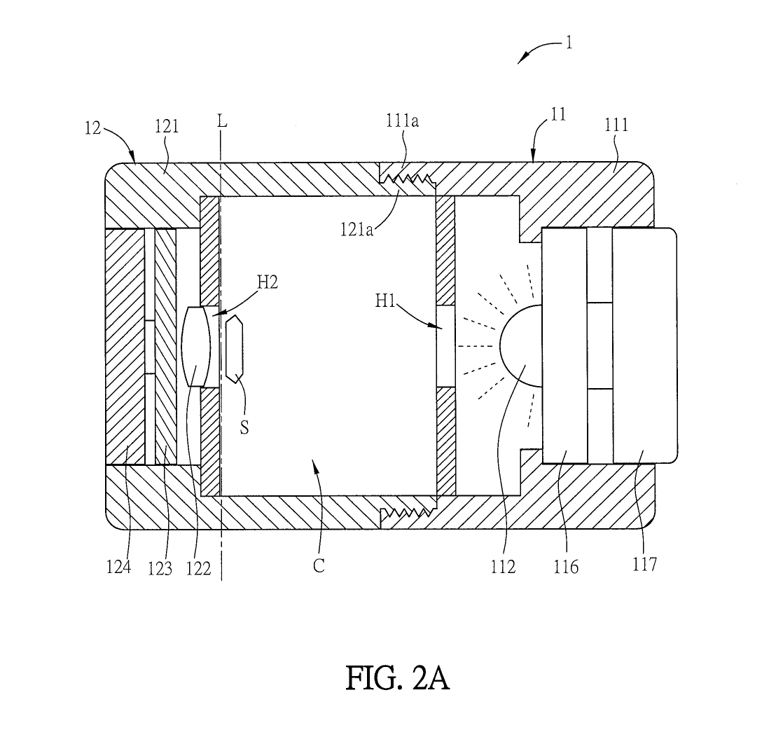

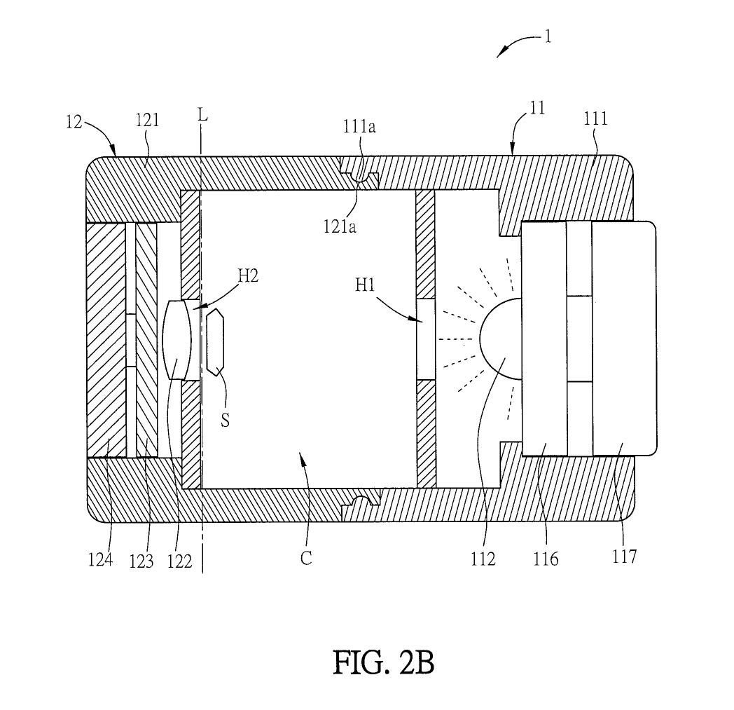

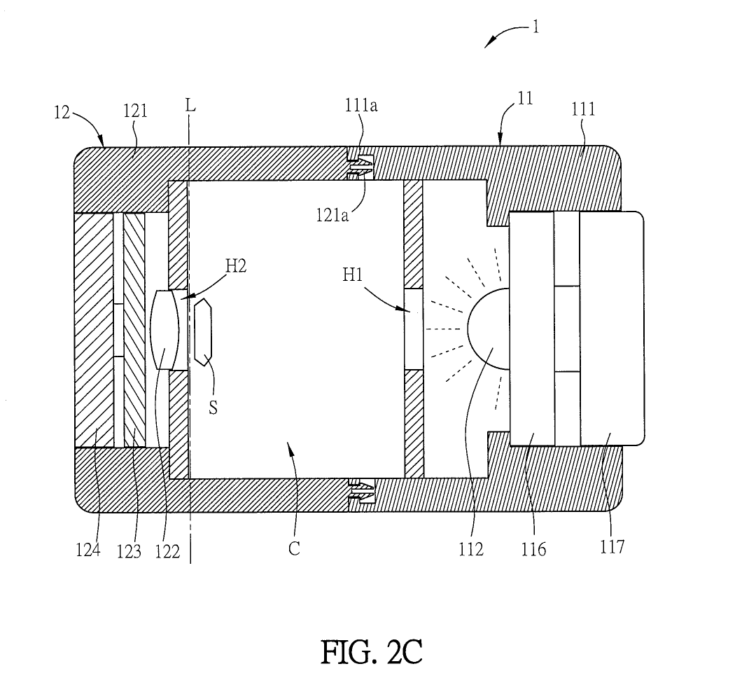

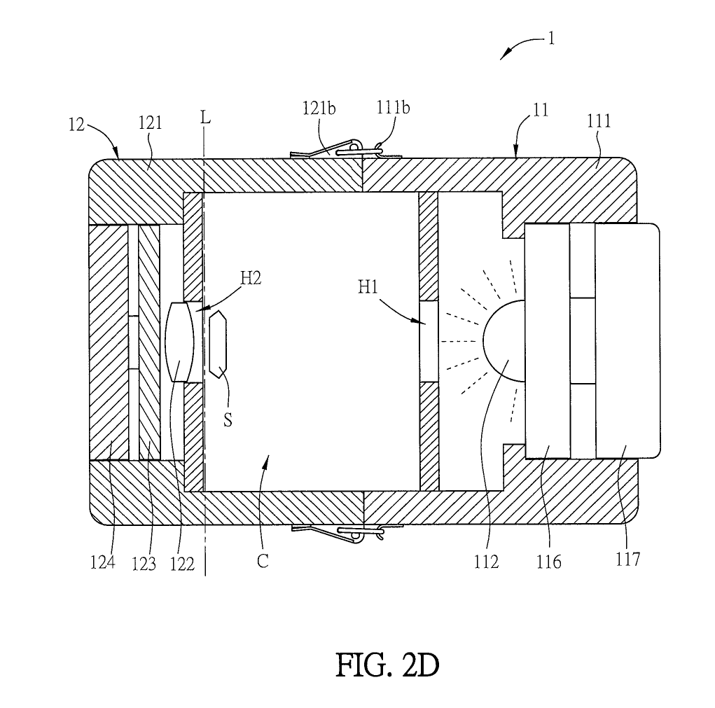

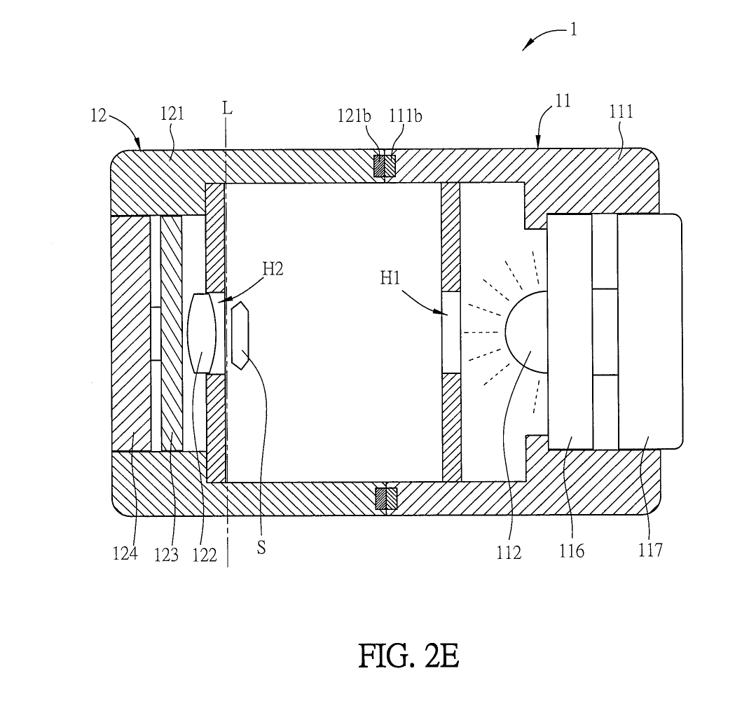

FIG. 1A is a schematic diagram showing the appearance of the assembled optical viewing device of an embodiment of the invention. FIG. 1B is a schematic diagram of the exploded perspective view of the optical viewing device in FIG. 1A. FIG. 2A is a schematic sectional diagram of the optical viewing device of FIG. 1A taken along the line A-A. FIGS. 2B to 2E are schematic diagrams of different embodiments of the optical viewing device of FIG. 2A. As shown in FIGS. 1A to 2E, the optical viewing device 1 can be used in cooperation with an external electronic device or a cloud server. The external electronic device can be a cell phone, a notebook computer, a tablet computer, a TV or a desktop computer. The cloud server refers to the server which can provide cloud computation or storage service. As shown in FIGS. 1 to 3, the optical viewing device 1 includes a first assembly 11 and a second assembly 12. The assembly refers to an object which is composed of a plurality of units, modules or elements to have a part of the function.

The first assembly 11 includes a first body 111, a light source 112, a battery 116 and a switch 117. The light source 112 is disposed within the first body 111 and includes at least a visible light source or at least an invisible light source. The switch 117 is disposed on a side of the first body 111 and electrically connected between the light source 112 and the battery 116 for switching the light source 112 to a turn-on state or a turn-off state. The battery 116 is disposed within the first body 111. The first body 111 includes a first connecting portion 111a and a light output hole H1, and the first connecting portion 111a is disposed on at least one side of the light output hole H1. As shown in FIGS. 2A to 2C, in this embodiment, the first connecting portion 111a is disposed around the light output hole H1.

The second assembly 12 includes a second body 121, a convex lens 122, an image capturing module 123 and a communication module 124. The second body 121 includes a second connecting portion 121a, which is disposed corresponding to the first connecting portion 111a. In this embodiment, the first connecting portion 111a and the second connecting portion 121a are a pair of matching thread structures which can be screwed and connected with each other, so that the first body 111 and the second body 121 can be fixed and connected to each other to form a chamber C. The sample S can be tissue section, blood, semen, cell culture liquid or tissue fluid, and is disposed in the chamber C. The convex lens 122 is disposed within the second body 121. The image capturing module 123 is disposed within the second body 121. The second body 121 includes a light input hole H2, and the second connecting portion 121a is disposed around the light input hole H2. The light source 112 provides a backlight source for the sample S. The light emitted by the light source 112 sequentially passes through the light output hole H1 and the chamber C and then diffuses in the sample and passes through the sample. Then, the light sequentially passes through the light input hole H2 and the convex lens 122 for the imaging. Subsequently, the image capturing module 123 captures the image formed by the convex lens 122 into a digital picture. The communication module 124 can be a wireless communication module (ex. WiFi or Bluetooth) or a wired communication module (a transmission cable), and is coupled with the image capturing module 123 to transmit the image signal from the image capturing module 123 to the external electronic device or the cloud server for the retouch, analysis, storage or development.

However, the above embodiment is just for the illustration but not for limiting the scope of this invention. As long as the first body 111 and the second body 121 can be fixed and connected to each other by the first connecting portion 111a and the second connecting portion 121a to form the chamber C for containing the sample S, the first connecting portion 111a and the second connecting portion 121a can be connected to each other by other ways. Some examples are provided as shown in FIGS. 2B and 2C. As shown in FIG. 2B, the first connecting portion 111a and the second connecting portion 121a are a pair of matching tenon structures and they can be connected to each other by tenon. In another embodiment, as shown in FIG. 2C, the first connecting portion 111a and the second connecting portion 121a are a pair of matching concave-convex structures and they can be connected to each other thereby.

In another embodiment, the first body 111 includes a first connecting element 111b and the second body 121 includes a second connecting element 121b. The first connecting element 111b and the second connecting element 121b are a pair of matching connecting elements. Some examples are provided as shown in FIGS. 2D and 2E. As shown in FIG. 2D, the first connecting element 111b and the second connecting element 121b are a pair of matching locking elements which can be locked and connected to each other. In FIG. 2E, the first connecting element 111b and the second connecting element 121b are a pair of magnets and they can be connected to each other by the magnetic attraction.

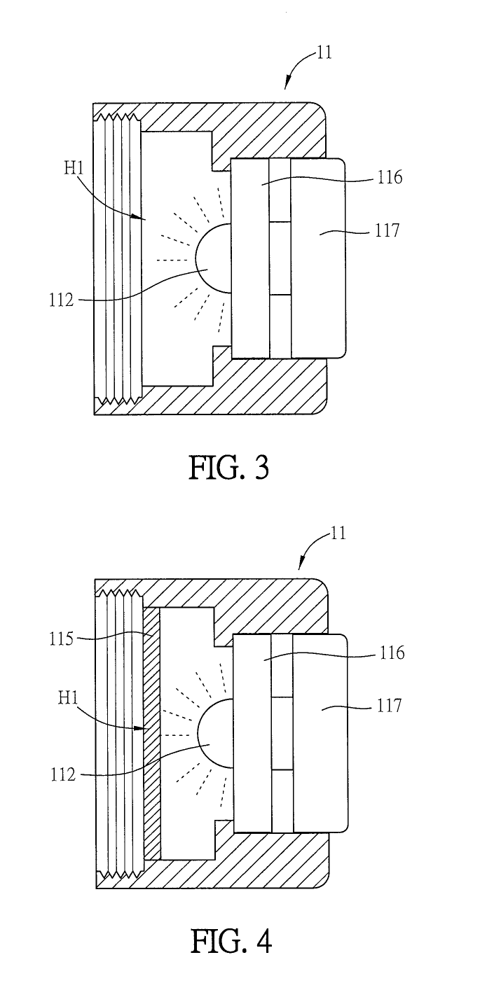

To be noted, in the above embodiments, the light output hole H1 is a hole on the first body 111 and the light source 112 is embedded within the first body 111 in a half-open type, but this invention is not limited thereto. For example, the light source 112 also can be disposed within the first body 111 in a fully-open type and the light output hole H1 is an opening of the first body 111 (as shown in FIG. 3). In another embodiment, in order to make the light emitted by the light source 112 evenly leave the light output hole H1, the first body 111 can further include a diffusion sheet 115 (as shown in FIG. 4) disposed on the light output hole H1. However, the above embodiment is just for the illustration but not for limiting the scope of the invention. As long as the light emitted by the light source can pass through the light output hole H1 and the chamber C and then enter the light input hole H2, the adjustment of the size or shape of the light output hole by those skilled in the art should belong to the scope of this invention.

By taking a solid sample S as an example, the operation of the optical viewing device 1 includes the steps of: disposing the sample S into the second assembly 12; connecting the first assembly 11 and the second assembly 12; vertically disposing the optical viewing device 1 as the second assembly 12 is on the bottom so that the sample S is located at the bottom of the chamber C, i.e. the focus plane L of the convex lens 122. To be noted, due to the digital/optical zoom function of the external electronic device, the focus plane L can be a region instead of a fixed plane, so that the sample S can be surely imaged accurately.

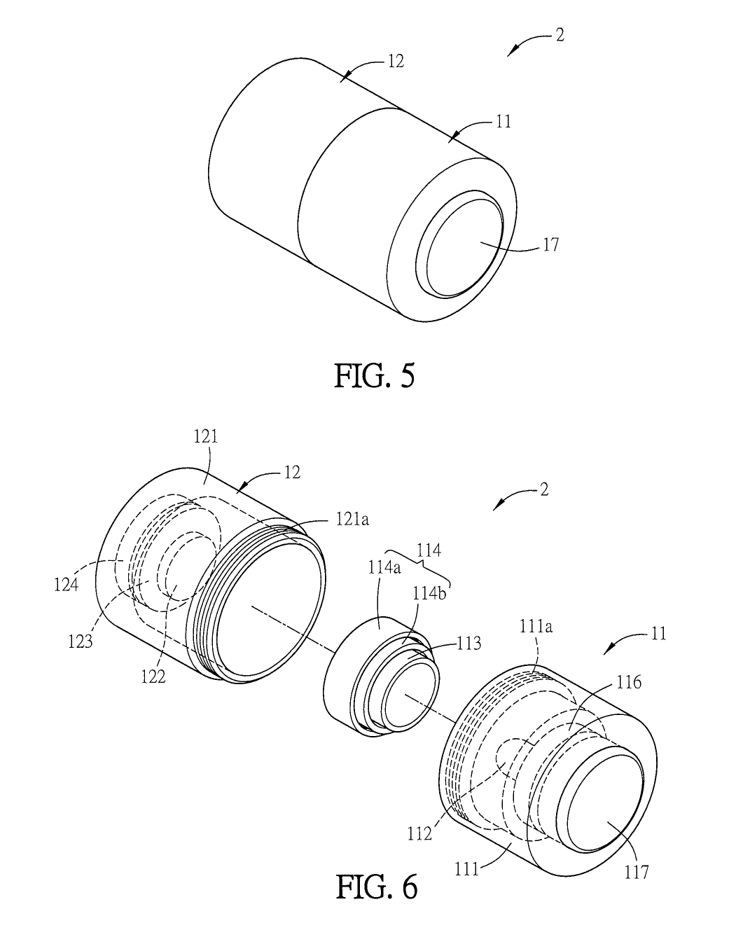

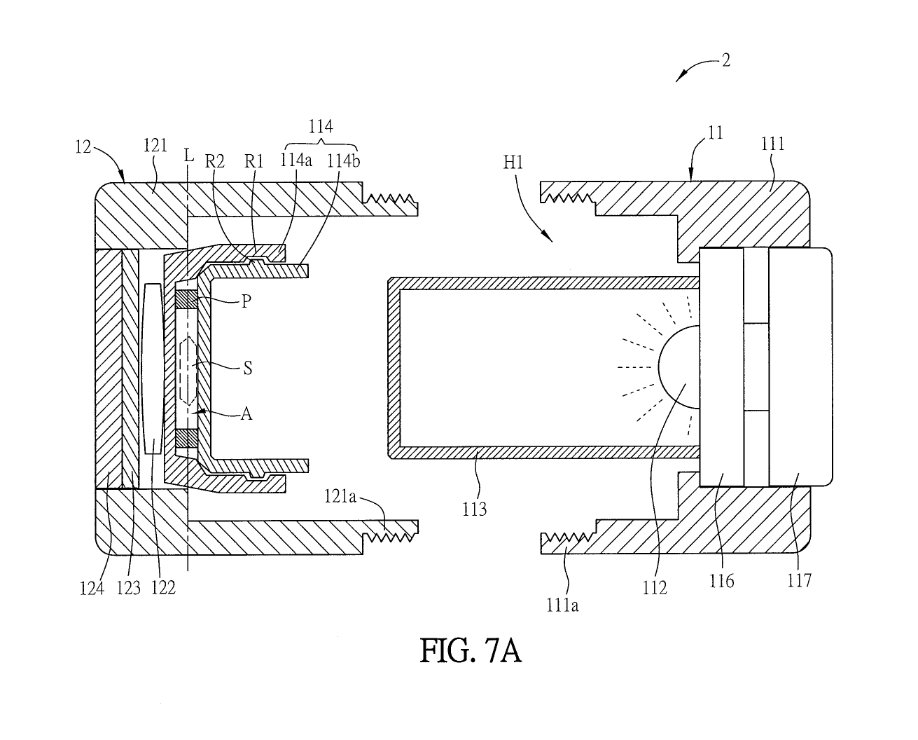

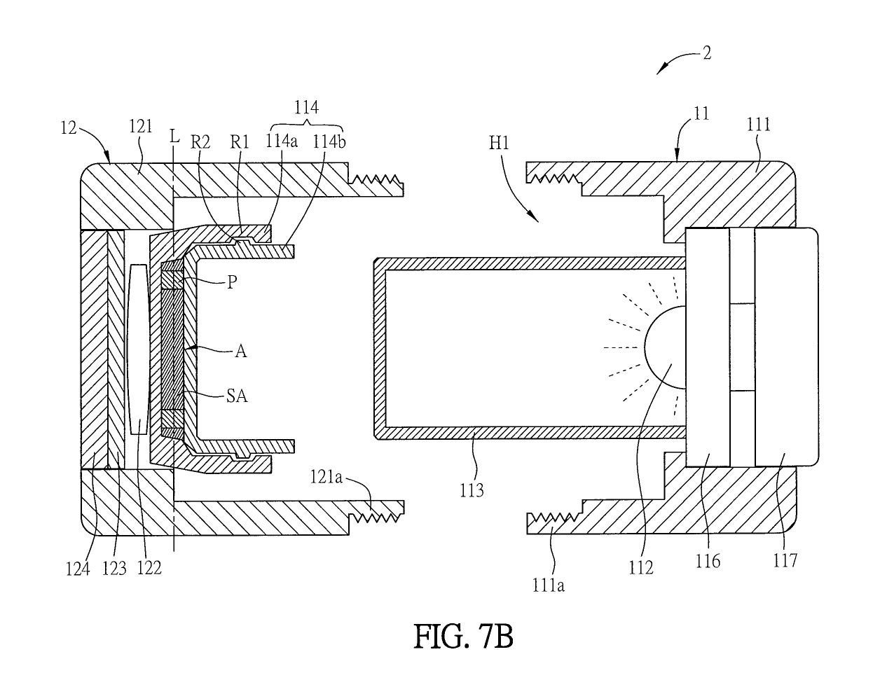

FIG. 5 is a schematic diagram showing the appearance of the assembled optical viewing device of another embodiment of the invention. FIG. 6 is a schematic exploded diagram of the optical viewing device in FIG. 5. FIGS. 7A and 7B are schematic sectional diagrams of the optical viewing device of FIG. 5 taken along the line A-A. As shown in FIGS. 5 to 7B, the composition of the optical viewing device 2 is approximately similar to the optical viewing device 1. The main difference between them is that the first assembly 21 of the optical viewing device 2 further includes a light guiding element 113 and a sampling assembly 114. The light guiding element 113 is disposed at the light output hole H1 and at least partially covers the light output hole H1 to receive the light emitted from the light output hole H1 and converge the light to the sample S. The light guiding element 113 can be a hollow housing or a solid transparent material. The sampling assembly 114 includes a first cup element 114a, a second cup element 114b and at least a positioning portion P. The first cup element 114a includes a first fixing structure R1 disposed around the wall of the first cup element 114a. In this embodiment, the first fixing structure R1 is a protruding ring, and the first cup element 114a covers the light guiding element 113. The second cup element 114b includes a second fixing structure R2 disposed corresponding to the first fixing structure R1. The second cup element 114b covers the outside of the first cup element 114a to collectively form a sample containing space A. In this embodiment, the positioning portion P is a small block structure disposed in the sample containing space A and coupled with the first cup element 114a or the second cup element 114b, so that the sample containing space A can be surely formed when the second cup element 114b covers the first cup element 114a because the bottoms thereof won't be tightly connected together. Besides, disposing the positioning portion P in the sample containing space A also can quantify the sample containing space A, so that the sample S within the sample containing space A can have a fixed observation thickness and therefore the light can pass through the sample and the sample can be located accurately on the focus plane L. The first fixing structure R1 and the second fixing structure R2 engage with each other to fix the first cup element 114a and the second cup element 114b relatively.

To be noted, the structural form of the positioning portion P and the fixing way between the first cup element 114a and the second cup element 114b mentioned in the above are just for the illustration but not for limiting the scope of this invention. The variation done by those skilled in the art after reading the structural form of the positioning portion P and the fixing way between the first cup element 114a and the second cup element 114b mentioned in the above should belong to the scope of this invention.

As shown in FIG. 7B, by taking the fluid sample SA as an example, the operation of the optical viewing device 2 includes the steps of: covering the light guiding element 113 by the first cup element 114a; wetting the outer surface of the first cup element 114a with the fluid sample SA; covering the first cup element 114a by the second cup element 114b with a slight pressing until they are locked with each other; connecting the first assembly 11 and the second assembly 12. To be noted, a gap is reserved between the first cup element 114a and the second cup element 114b to allow the overflow of the fluid sample SA. Therefore, the fluid sample SA is positioned and quantitatively disposed on the focus plane L of the convex lens 122 through the sampling assembly 114, so that the fluid sample SA can be surely imaged accurately.

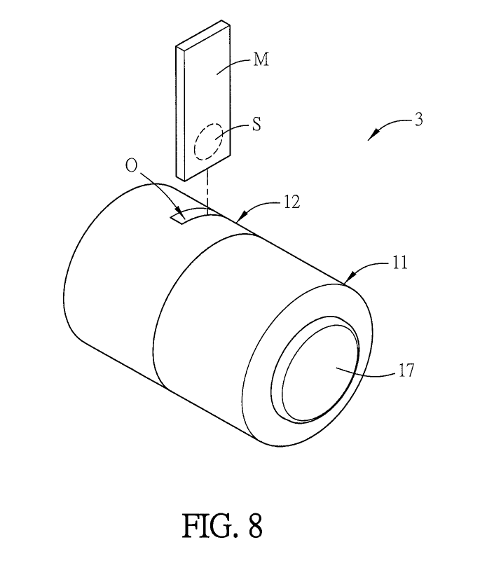

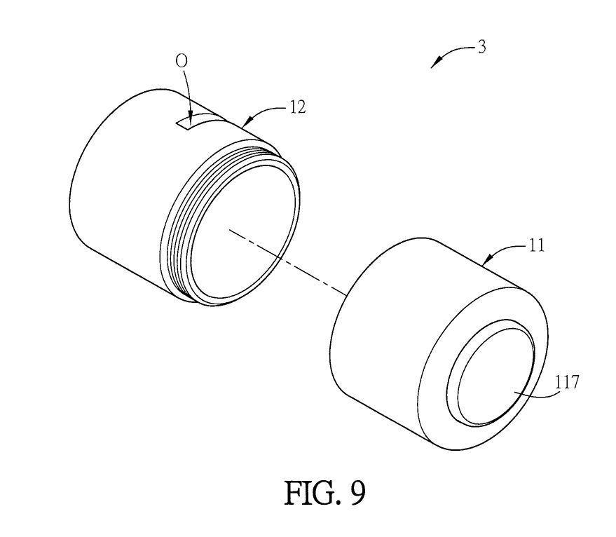

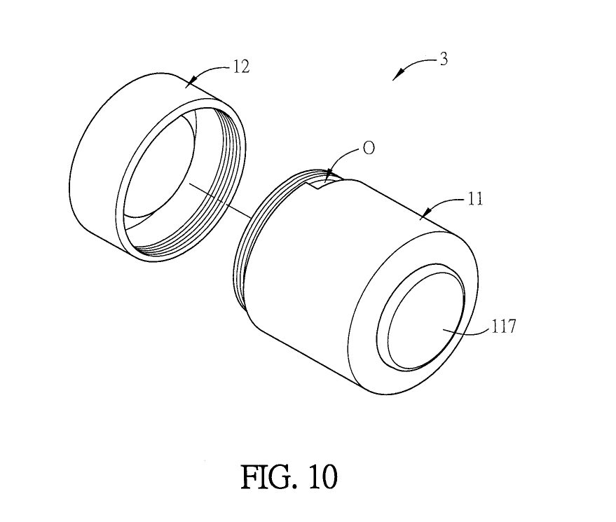

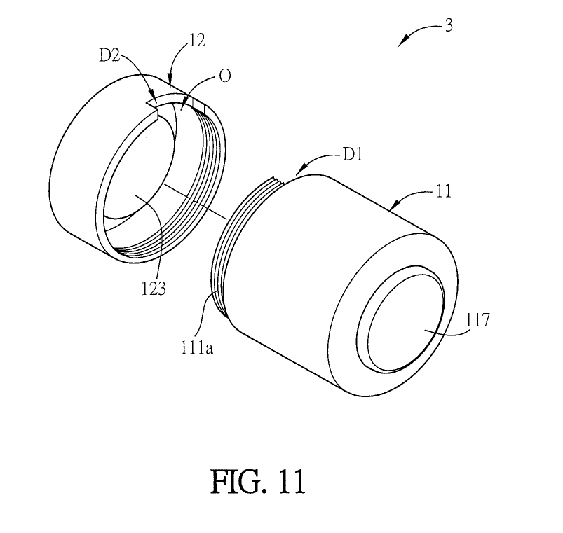

FIG. 8 is a schematic diagram showing the appearance of the optical viewing device of another embodiment of the invention. FIG. 9 is a schematic exploded diagram of the optical viewing device in FIG. 8. FIG. 10 is a schematic exploded diagram of another embodiment of the optical viewing device in FIG. 8. FIG. 11 is a schematic exploded diagram of another embodiment of the optical viewing device in FIG. 8. As shown in FIGS. 8 to 11, the composition of the optical viewing device 3 is approximately similar to the optical viewing device 1. The main difference between them is that the chamber C of the optical viewing device 3 includes an opening O (as shown in FIG. 9). A carrier M carrying the sample S matches the opening O in shape and size, so that the carrier M can enter the chamber C (not shown) through the opening O for the observation. The carrier M can be a dropper or a slide. To be noted, the opening O is disposed corresponding to the focus plane L (not shown) of the convex lens 122, so that the sample S can be located on the focus plane L of the convex lens 122 when entering the chamber C through the carrier M. Moreover, in this embodiment, the opening O can be disposed on the first connecting portion 111a (not shown), but this invention, however, is not limited thereto. In another embodiment, the opening O can be disposed on the second connecting portion 121a. In another embodiment, the opening O can be partially disposed on the first connecting portion 111a and partially disposed on the second connecting portion 121a (as shown in FIG. 11). In other words, the first connecting portion 111a has a first breach D1, the second connecting portion 121a has a second breach D2, and the first breach D1 and the second breach D2 match each other and form the opening O after connecting to each other.

In summary, the optical viewing device of this invention can largely lower down the technological threshold of the sample detection, so that the user untrained professionally also can easily perform the sample detection. Therefore, even the unprofessional operator can perform the operation of the optical viewing device at the sampling site. Thus, the detection process can be simplified, the burden of the detection station can be lowered down, and the freshest sample can be surely viewed.

Although the invention has been described with reference to specific embodiments, this description is not meant to be construed in a limiting sense. Various modifications of the disclosed embodiments, as well as alternative embodiments, will be apparent to persons skilled in the art. It is, therefore, contemplated that the appended claims will cover all modifications that fall within the true scope of the invention.

What is claimed is:

1. An optical viewing device, comprising:
a first assembly comprising:
a first body comprising a first connecting portion and a light output hole, wherein the first connecting portion is disposed on at least one side of the light output hole, and
a light source disposed within the first body; and
a second assembly comprising:
a second body comprising a second connecting portion, which is disposed corresponding to the first connecting portion,
a convex lens disposed within the second body, and
an image capturing module disposed within the second body, wherein the light outputted from the light output hole passes through the convex lens and then enters the image capturing module;
wherein the first connecting portion and the second connecting portion match and connect each other to form a chamber.

2. The optical viewing device as recited in claim 1, wherein the first connecting portion is disposed around the light output hole.

3. The optical viewing device as recited in claim 1, wherein the second body comprises a light input hole, the second connecting portion is disposed around the light input hole, and the light emitted by the light source sequentially passes through the light output hole, the chamber and the light input hole.

4. The optical viewing device as recited in claim 1, wherein the first connecting portion and the second connecting portion are connected to each other by screw or tenon.

5. The optical viewing device as recited in claim 1, wherein the chamber comprises an opening and the opening matches a carrier.

6. The optical viewing device as recited in claim 5, wherein the opening is disposed on the first body or the second body.

7. The optical viewing device as recited in claim 6, wherein the first body comprises a first breach, the second body comprises a second breach, and the first breach and the second breach match and connect each other to form the opening.

8. The optical viewing device as recited in claim 1, wherein the first assembly further comprises:
a light guiding element disposed at the light output hole, wherein the light emitted by the light source sequentially passes through the light output hole, the light guiding element and the chamber.

9. The optical viewing device as recited in claim 8, wherein the first assembly further comprises:
a sampling assembly covering the light guiding element.

10. The optical viewing device as recited in claim 9, wherein the sampling assembly comprises:
a first cup element covering the light guiding element; and
a second cup element covering an outside of the first cup element;
wherein the first cup element and the second cup element are connected to each other in a concave-convex match to form a sample containing space.

11. The optical viewing device as recited in claim 10, wherein the sampling assembly further comprises:
at least a positioning portion disposed in the sample containing space.

12. An optical viewing device, comprising:
a first assembly comprising:
a first body comprising a first connecting element and a light output hole, wherein the first connecting element is disposed on at least one side of the light output hole, and
a light source disposed within the first body; and
a second assembly comprising:
a second body comprising a second connecting element, which is disposed corresponding to the first connecting element,
a convex lens disposed within the second body, and
an image capturing module disposed within the second body;
wherein the first connecting element and the second connecting element match and connect each other to form a chamber, and the light outputted from the light output hole sequentially passes through the light output hole, the chamber and the convex lens and then enters the image capturing module.

13. The optical viewing device as recited in claim 12, wherein the first connecting element and the second connecting element are connected to each other by locking or magnetic attraction.

14. The optical viewing device as recited in claim 12, wherein the chamber comprises an opening and the opening matches a carrier.

15. The optical viewing device as recited in claim 14, wherein the opening is disposed on the first body or the second body.

16. The optical viewing device as recited in claim 14, wherein the first body comprises a first breach, the second body comprises a second breach, and the first breach and the second breach match and connect each other to form the opening.

17. The optical viewing device as recited in claim 12, wherein the first assembly further comprises:
a light guiding element disposed at the light output hole, wherein the light emitted by the light source sequentially passes through the light output hole, the light guiding element and the chamber.

18. The optical viewing device as recited in claim 17, wherein the first assembly further comprises:
a sampling assembly covering the light guiding element.

19. The optical viewing device as recited in claim 18, wherein the sampling assembly comprises:
a first cup element covering the light guiding element; and
a second cup element covering an outside of the first cup element;
wherein the first cup element and the second cup element are connected to each other in a concave-convex match to form a sample containing space.

20. The optical viewing device as recited in claim 19, wherein the sampling assembly further comprises:
at least a positioning portion disposed in the sample containing space.
